(19) United States
(12) Patent Application Publication
(10) Pub. No.: US 2004/0243039 A1
(43) Pub. Date: Dec. 2, 2004

(54) Cybernetic spine stretching table

(76) Inventors: Anthony Heng, Melun (FR); Pierre Bompard, Versailles (FR)

Correspondence Address: Roger W. Parkhurst, Parkhurst & Wendel, 1421 Prince Street, Suite 210, Alexandria, VA, 22314-2805, US

(21) Appl. No.: 10/488,524

(22) PCT Filed: Sep. 11, 2002
(86) PCT No.: PCT/FR02/03099

(30) Foreign Application Priority Data
Sep. 11, 2001 (FR) 01/11717

(51) Int. Cl. A61F 5/00

(52) U.S. Cl. 602/32

(57) ABSTRACT

The invention concerns a device to stretch the spinal and thoracic muscles of a patient composed of a table (100) with substantially horizontal reception surface on which to rest a patient's hips and shoulders, immobilizing means applied to the back of the patient's lower limbs and preventing them from moving, a stretching means being able to slide, be immobilized in position and/or exert an effort consisting in distancing the patient's hands from the joining of thighs and hips through a winch or a motor reeling cables (7 and 8) linked to the ends of the support. A second winch adjust the tension of the cable of the knees (9). A rolling headrest on the support enables to stretch the neck muscles when the handle (15) clamps the brake (16) which blocks the rule (17) linking it to the carriage (4). The system assembly provides what may be called cybernetic stretching of the spine whereof the force is selected by the user at all times on the basis of the reaction of his muscles. The device is designed for treating spinal pains and deformations.

The technical scope of the present invention is that of stretching systems for the spine of a patient so as to release handicapping contractions.

The purpose of the system is thus to stretch the spinal and thoracic muscles whose retraction causes pain and strain.

The usual devices are traction tables that apply an external force, the intensity of which determined by the therapist is peremptory for the user's muscles, to the two ends of the user's trunk through the action of a motor. This causes poor results and accidents and requires the mandatory presence of a therapist.

Other known devices use the patient's own weight by way of a stretching force thanks to inclined or suspended plane systems on which the patient is positioned. One of them uses in addition the pushing force of the arms applied along the body onto grips fastened at the hips. The intensity of the force applied is, once again, not sufficiently modifiable and the possible postures are unequivocal.

Other known devices allow isotonic work on the muscles consisting of a succession of muscular contractions mobilizing the spinal muscles with a starting position that ensures a certain spinal prevention. These devices are rather more designed to increase musculature than to stretch the spine.

The system according to the invention overcomes these insufficiencies by proposing a device only allowing the spine of a patient to be stretched.

The invention thus relates to a device to stretch the spinal and thoracic muscles of a patient composed of a table on which the trunk is positioned substantially horizontally, and a traction system, wherein it incorporates:

a substantially horizontal reception surface on which to rest a patient's hips and shoulders,

immobilizing means applied to the back of the patient's lower limbs and preventing them from moving,

stretching means located in the cranial prolongation of the patient's trunk, which the patient may grip onto, these means being able to slide, be immobilized in position and/or exert an effort consisting in distancing the patient's hands from the joining of thighs and hips.

According to one characteristic, the device incorporates a support cushion on which the patient's hips rest.

According to another characteristic, the device comprises a roller positioned behind the patient's lower limbs to keep the patient's thighs bent onto his stomach and to exert a bending stress on the thighs.

According to yet another characteristic, the thickness of the cushion and the angle formed by the faces on which respectively the patient's stomach and thighs rest are modifiable, in particular by the addition of extra cushions that are triangular and/or parallelepipedic in shape and held in place by adhesives.

According to yet another characteristic, the cushion has a median excavation in its lower face at the bottom of which a fastening element is attached for a link such as a strap connecting it to the roller.

According to yet another characteristic, the support surface is limited by a rear cushion and a front cushion, the distance between which can be adjusted by moving the rear cushion.

According to a variant embodiment, the stretching means are constituted by:

a support fastened to the table by means of a beam,

a carriage able to slide on the support,

a transverse bar connected to the carriage and fitted at both ends with a handle which the patient holds,

drive means able to control the immobilization of the carriage and/or its displacement to obtain a stretching effect on the patient's spine.

According to one characteristic, the drive means are constituted by a pawl winch concomitantly winding and unwinding the two cables fastened to the ends of the support, this winch being activated by two levers onto which the bar is fastened, such that the patient is able to activate the winch in traction by pulling on the bar and lessen the traction by pushing on the bar.

According to yet another characteristic, the roller is retained and moved forwards along the table by a cable wound round a pawl winch that the patient is able to activate thanks to a handle on the carriage.

According to another variant, the stretching means are formed of:

a support fixed under the front part of the table by means of a beam,

a carriage sliding on the support,

a bar connected to the carriage and fitted at both ends with handles onto which the patient holds

drive means able to control the immobilization of the carriage and/or the stretching load, these means being a motor, fastened onto the carriage, concomitantly winding and unwinding two cables one of which is fastened to the table and the other slides at one end of the support and is fastened to the roller, this motor being activated by a button located on one of the handles such that the patient is able to activate the traction.

According to one characteristic, the device incorporates means to position the head and stretch the neck muscles, this system integrating a head rest integral with a rule sliding on the carriage and likely to be integral in translation with the rule by means of a brake.

According to yet another characteristic, the head rest incorporates two rounded pads, adjustable in height and spacing, on which the cheeks rest, and on the edges of which straps are fastened to one another by adhesives.

According to yet another characteristic, the support may be inclined from the horizontal position to an angle of around 60° downwards.

Other characteristics, particulars and advantages of the invention will become more apparent from the following description, given hereafter by way of illustration and in reference to the appended drawings, in which:

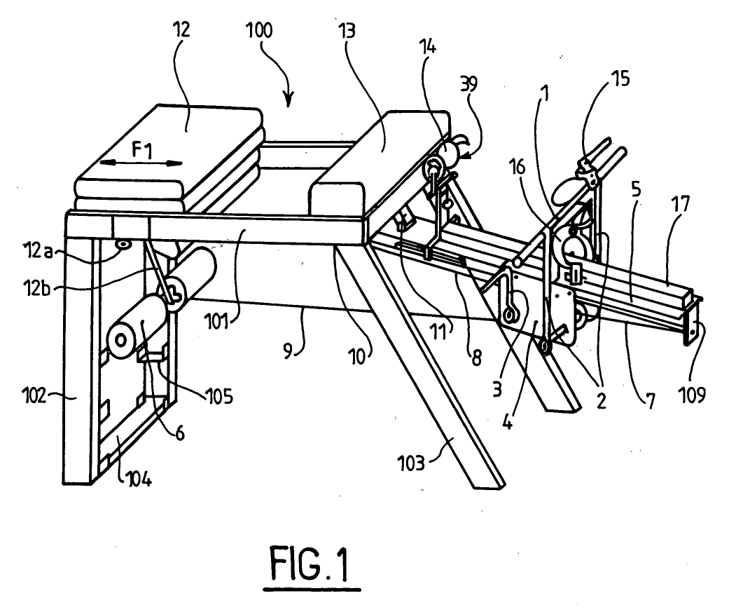

FIG. 1 is a profile view of the device according to the invention according to a first embodiment,

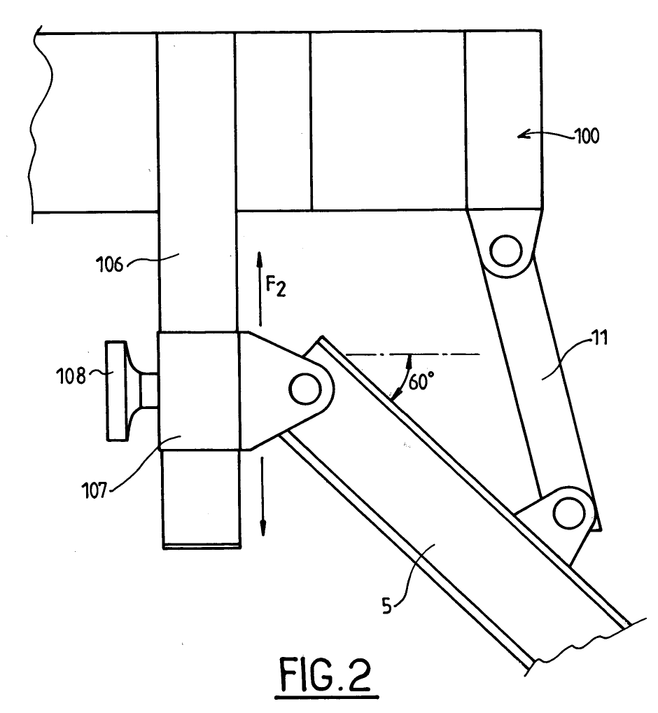

FIG. 2 is a section showing the fastening of the carriage on the table,

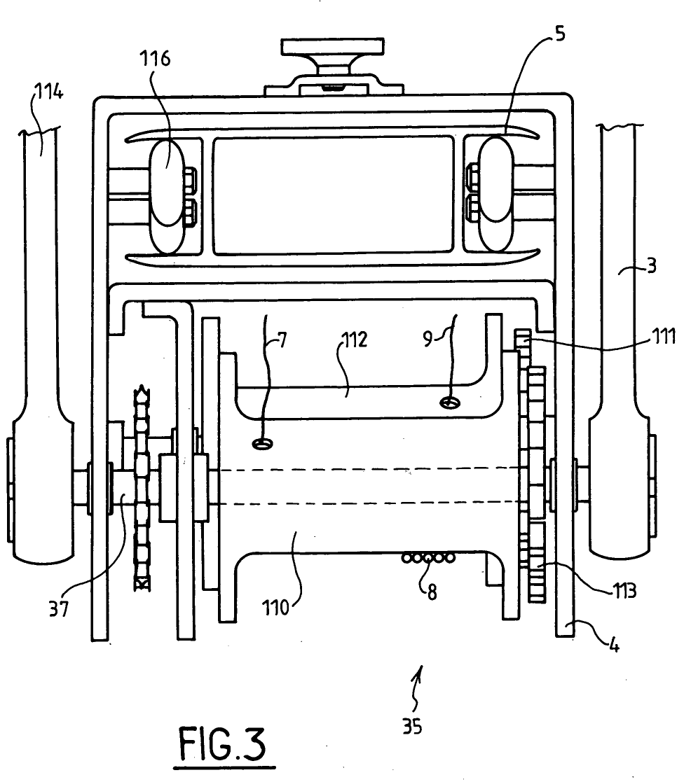

FIG. 3 is a section showing a first embodiment of the drive means,

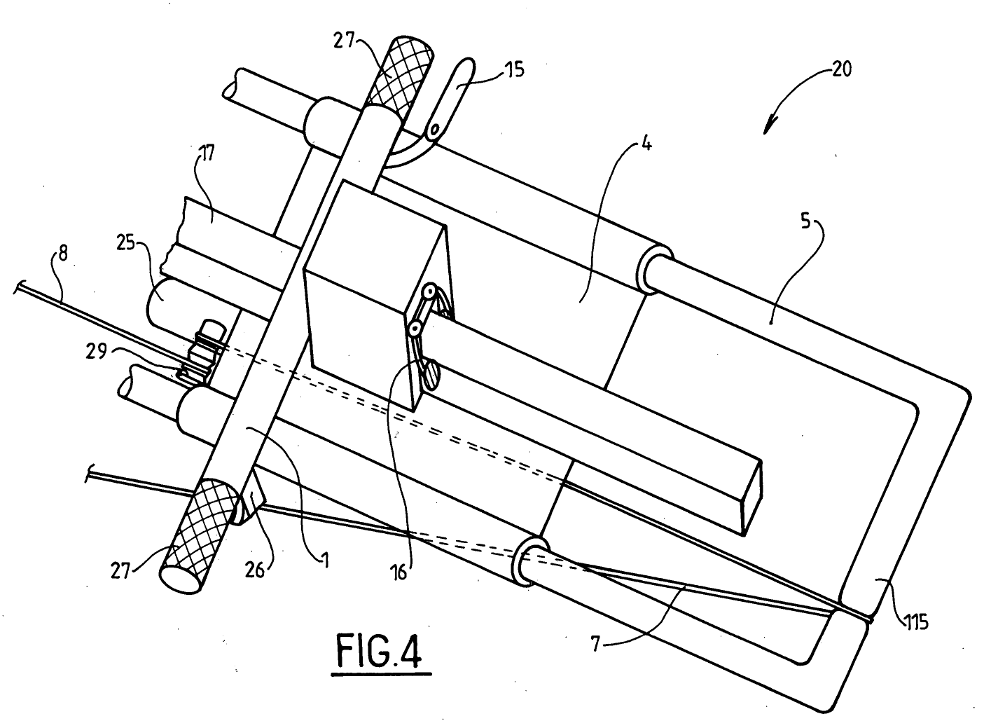

FIG. 4 is a top view of the invention according to a second embodiment,

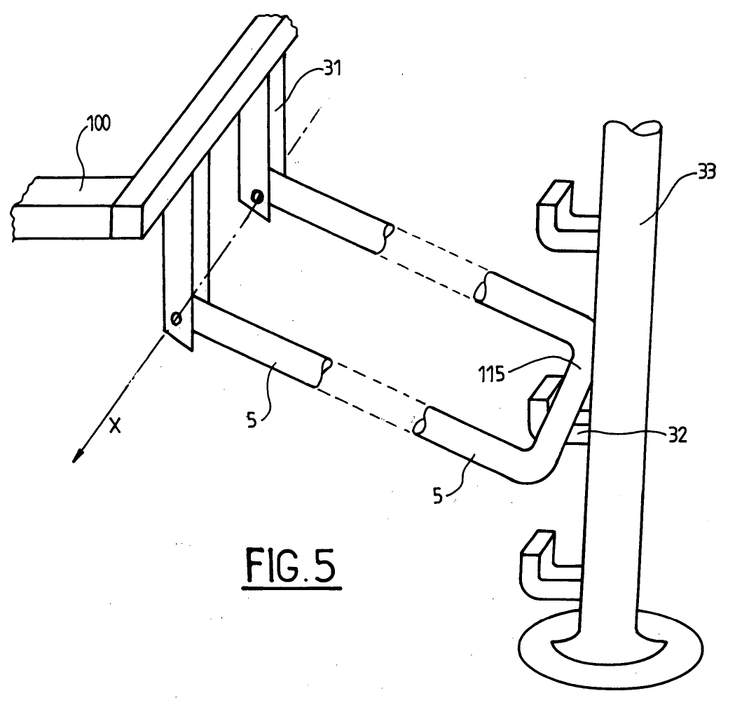

FIG. 5 is a view illustrating an embodiment of the tilting of the support, and

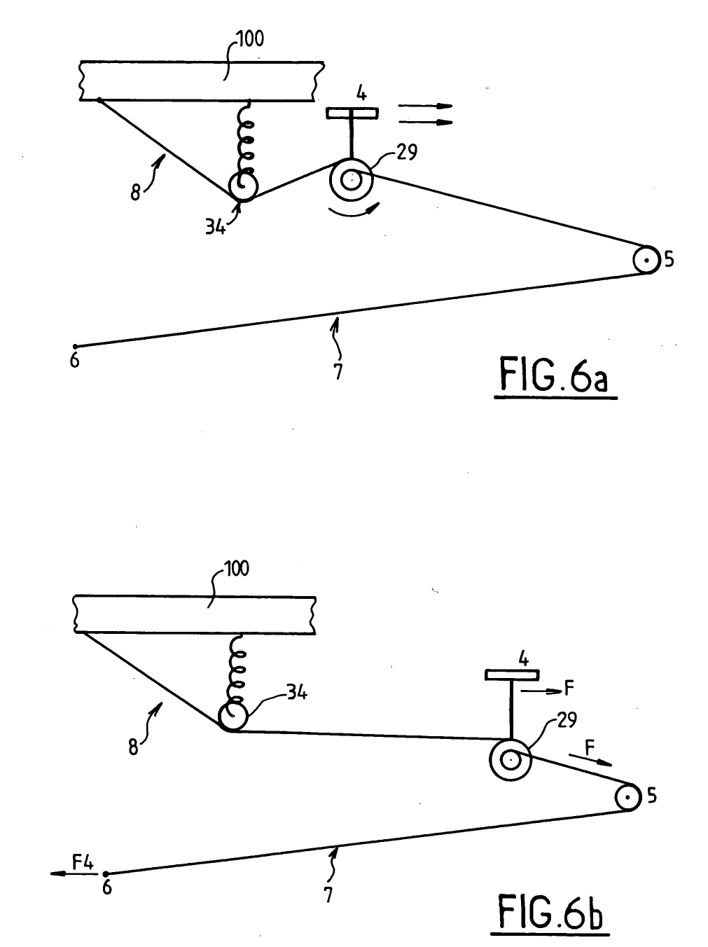

FIGS. 6a and 6b are schemas illustrating the mode of displacement of the carriage.

FIG. 1 shows a table 100 formed of a frame 101 resting on two front legs 102 and two back legs 103. The two back legs 102 may be joined by a cross piece 104 so as to make the assembly more rigid. The table 100 incorporates two cushions 12 and 13 constituting a support for the patient's body. The front cushion 13 is fixed and intended for the patient's shoulders, the other 12, to the rear, may slide backwards and forwards following arrow F1 in order to adapt to the patient's height and is immobilized in the require position by a roller wheel 12a. The rear cushion 12, whose planes form a posterior vertex, is padded on the two faces that are in contact with the patient.

It can be adjusted both in the angle between its upper and lower faces and in the thickness of its posterior edge such that it can be adapted to the angle selected by the user between the plane of the stomach and that of the thighs. The height is adjusted by adding extra parallelepipedic or triangular-shaped cushions held together by adhesive straps, not shown.

The lower face may have a median excavation (not shown) to free access to the solid structure of the cushion 12 at which point a fastening for the suspension strap 12b of a roller 6 is fixed, such roller being intended to be applied to the rear of the lower limbs.

The front legs 103 of the table may be inclined in order to balance the assembly. The rear legs 102 have staggered supports 105 to provide a mobile scale allowing children to use the table according to the invention. The dimensions of the table are, for example, such that the feet of an adult patient of average height installed on it touch the floor.

The table 100 is extended forwards by a support 5 of an elongated shape. This support is fastened onto the table on a lower level than the support surface 13. This support receives a head rest 14 and a mobile carriage 4. It may be in the form of a rail, as seen in FIG. 1, or in the form of a frame, as seen in FIG. 4.

The support 5 incorporates two running treads onto which four pairs of castors 116 from the upper part of the carriage 4 are applied as are four castors from the head rest 14. At both ends, the support has an abutment 109 to fasten the cable 7 connected to the fore of the carriage 4. The cable 8 connects the carriage 4 to the table 100.

A cable 9 connects the carriage 4 to the roller 6.

The carriage 4 is designed so as to transform the alternative traction movements of the patient's arms on the bar 1 into a forwards movement.

In its lower part at the front, the carriage 4 comprises two pawl levers 2 on either side, welded to the bar 1 and intended for the hands. These levers activate the coil of a winch 35, shown in FIG. 3 and onto which the two cables 7 and 8 fastened to the ends of the support concomitantly wind and unwind. This winch 35 constitutes drive means. FIG. 3 shows the carriage 4, the support 5 and the drive means 35. These two pawl levers 2 can be set in two positions: “traction” and “release”. The coil 110 has a toothed wheel 111 and a spring-loaded pawl blocking its rotation in a clockwise direction. The anti-return wheel may be released by a lever (not shown) located on the hidden side of the carriage 4. Thus, the carriage does not move when the bar 1 is pushed forwards, but moves forwards when the bar 1 is pulled by the user.

In its lower part at the rear, a second winch 112 is placed with its toothed wheel 113 and its spring-loaded pawl, and activated by a third pawl lever 3. This third pawl lever can be set in two positions: “traction” and “release”. The toothed wheel 113 can be released by a lever 114 located on the hidden side of the carriage. This winch 112 winds the cable 9 of the bar 1 intended for the back of the patient's knees. The anti-return system of the winch blocks the rearward movement of the knee roller.

The head rest 14 completes the device shown in FIG. 1. It is formed of two pads upon which the cheeks are intended to rest and to which straps (not shown) are fastened intended to be attached to the nape of the patient's neck. The head rest can be adjusted in the spacing of the pads and in its height and tilt thanks to control knobs in a known manner. The four castors fixed to its base allow it to roll freely on the support 5. A brake 16 placed on the upper face of the carriage by tightening a rule 17 fixed on its lower part makes it integral with the movements of the carriage when the hand presses the handles 15 allowing the neck muscles to be stretched.

FIG. 2 shows means to adjust the downward tilt of the support 5 between a substantially horizontal position to an angle of around 60°, thanks to its hinged fastened on a beam 106 and to a rocker bar 11 fastened under the front part of the table 100. The beam 106 is provided with a sliding cap 107 onto which the carriage is fastened. The positioning of the cap 107 according to arrow F2 is fixed by means of a knurled bolt 108 to adjust the position of the support 5.

FIG. 4 shows a variant embodiment of the muscle stretching system. In this embodiment, the manual traction system is proposed to be replaced by an electric or pneumatic motor 35, the assembly being fixed onto a frame 5. The motor 25 is fixed to the frame 5 and its drive shaft supports a double pulley 29. The bar 1, which the patient holds, is integral with the carriage 4 and a control 26 for the motor is fixed to the bar 1 so that the patient is easily able to activate the motor 25. This control may, for example, be one or several buttons controlled the start up of the motor 25 in a traction direction or in the opposite direction. When the button is released, the motor 25 comes to an immediate halt, for safety reasons. The motor 25 drives the pulley 29, on which the cables 7 and 8 wind, in rotation. When the carriage 4 moves away from the table 100, the pulley 29 unwinds cable 8 and winds cable 7.

Such an arrangement allows the patient to very precisely adjust the displacement of the bar 1 and the tension applied to his muscles. The use of the button 26 allows the action of the motor 25 to be stopped instantly. Moreover, the system may easily be adapted, for example, by using the rotation of the handles 27 to control the motor.

In FIG. 4, the cable 7 is no longer fixed to the support 5 as explained in relation to FIG. 1 but passes in a groove made in the bar 115 of the frame 5 and is turned over so as to be attached to the roller 6.

Consequently, the device according to the invention allows a stretching operation to be made that does not require the use of any external force, as described in relation to FIG. 1, or that uses a motor to drive the carriage 4.

In the first case, the force used is that of the upper limbs of the user holding onto the carriage with his hands, and of the lever formed by the femurs being pulled forwards by the knees and pulling the hips backwards around the fixed point of the rear cushion. These forces are managed at all times by the user according to the sensations in his own muscles, and thus the stretching operation may be termed cybernetic. The resistance of the carriage to any traction of the arms and the knee bar to any pushing of the rear face of the thighs allows the previously stretched muscles to be subjected to a muscular contraction that gives this stretching operation a particular character known as myotensive.

This device allows the full stretching of the spinal muscles, including those of the cervical vertebrae whose position may be selected.

This device is safe since the subject is in a stable position on the table and the simple fact of releasing the bar stops any traction on the spine, keeping only the bended leg position that is easy to get out of thanks to the release lever on the anti-return system.

Lastly, this device is fully adjustable at all sizes.

In the second case, it is the motor 25 controlled by the patient which allows the carriage's displacement to be adjusted and therefore the intensity of the stretching.

FIG. 5 shows a second mode of adjusting the position of the support 5. The support 5 is held, at its first end, by two lugs 31, integral with the table 100, and can pivot with respect to these lugs around an axis X. The adjustment means are constituted by a leg 33 having different levers 32 over which the bar 115 is hooked.

FIGS. 6a and 6b are schemas illustrating the mode of displacement of the carriage 4.

As explained previously, the cable 8 is connected to the table 100 at one end and is wound on the double pulley 29 at its other end. The cable 7 is also wound on the pulley 29 at one end, passes in a groove machined into the support 5 (which can be seen in FIG. 4) and is then connected by its other end to the roller 6. A tension device 34 is provided to ensure the tension of the cable 8 and to modify its length according to the size of the patient.

When the pulley 29 turns anti-clockwise, driven by the motor 25, the cable 8 unwinds, whereas the cable 7 winds around the pulley. The carriage 4, onto which the pulley 29 is fastened, moves away from the table 100 and the tension exerted by the cable 7 pulls both the patient's hands and lower members forwards. This system allows the spinal muscles to be stretched, whilst bringing the patient's thighs closer to his stomach so as to amplify the stretch.

An attempt by the patient exerted in the direction of arrow F4 to push his legs away from his stomach, as show in FIG. 6b, tightens the cable 7, thereby further distancing the carriage 4 from the table 100 and further stretching the muscles.

The device according to the invention is particularly designed for physiotherapists and physical therapist.

1. A device to stretch the spinal and thoracic muscles of a patient composed of a table (100) on which the trunk is positioned substantially horizontally, and a traction system, wherein it incorporates:
a substantially horizontal reception surface (12, 13) on which to rest a patient's hips and shoulders,
immobilizing means (6) applied to the back of the patient's lower limbs and preventing them from moving,
stretching means (20) located in the cranial prolongation of the patient's trunk, which the patient may hold onto, these means being able to slide, be immobilized in position and/or exert an effort consisting in distancing the patient's hands from the joining of thighs and hips.

2. A device according to claim 1, wherein it incorporates a support cushion (12) on which the patient's hips rests.

3. A device according to claim 2, wherein it comprises a roller (6) positioned behind the patient's lower limbs to keep the patient's thighs bent over his stomach and to exert a bending stress on the thighs.

4. A device according to claim 3, wherein the thickness of the cushion (12) and the angle formed by the faces on which respectively the patient's stomach and thighs rest are modifiable, in particular by the addition of extra cushions that are triangular and/or parallelepipedic in shape and held in place by adhesives.

5. A device according to claim 4, wherein the cushion (12) has a median excavation in its lower face at the bottom of which a fastening element is attached for a link such as a strap connecting it to the roller (6).

6. A device according to claim 1, wherein the support surface is limited by a rear cushion (12) and a front cushion (13), the distance between which can be adjusted by moving the rear cushion (12).

7. A device according to claim 1, wherein the stretching means (20) are constituted by:
a support (5) fastened to the table (100) by means of a beam (21),
a carriage (4) able to slide on the support (5),
a transverse bar (1) connected to the carriage (4) and fitted at both ends with a handle (27) which the patient holds,
drive means (35) able to control the immobilization of the carriage (4) and/or its displacement to obtain a stretching effect on the patient's spine.

8. A device according to claims 7, wherein the drive means (35) are constituted by a pawl winch (36) concomitantly winding and unwinding the two cables (7, 8) fastened to the ends of the support (5), this winch (22) being activated by two levers (3, 38) onto which the bar (1) is fastened, such that the patient is able to activate the winch in traction by pulling on the bar (1) and lessen the traction by pushing on the bar (1).

9. A device according to claim 8, wherein the roller (6) is retained and moved forwards along the table (100) by a cable (9) wound round a pawl winch (23) that the patient is able to activate thanks to a handle (3) on the carriage (4).

10. A device according to claim 1, wherein the stretching means (20) are formed of:
a support (5) fixed under the front part of the table (100) by means of a beam (21),
a carriage (4) sliding on the support (5),
a bar (1) connected to the carriage (4) and fitted at both ends with handles (27) onto which the patient holds
drive means (25) able to control the immobilization of the carriage (4) and/or the stretching load, these means being a motor (25), fastened onto the carriage (4), concomitantly winding and unwinding two cables (7, 8) one of which is fastened to the table (100) and the other slides at one end of the support (5) and is fastened to the roller (6), this motor (25) being activated by a button (26) located on one of the handles (27) such that the patient is able to activate the traction.

11. A device according to claim 1, wherein it incorporates means (39) to position the head and stretch the neck muscles, this system integrating a head rest (14) integral with a rule (17) sliding on the carriage (4) and likely to be integral in translation with the rule (17) by means of a brake (16a).

12. A device according to claim 11, wherein the head rest (14) incorporates two rounded pads, adjustable in height and spacing, on which the cheeks rest, and on the edges of which straps are fastened to one another by adhesives.

13. A device according to claim 1, wherein the support (5) can be inclined from the horizontal position to an angle of around 60° downwards.
